(19) United States
(12) Patent Application Publication
(10) Pub. No.: US 2008/0125732 A1
(43) Pub. Date: May 29, 2008

(54) Potty pony pals training underpants

(76) Inventor: Doreen Rutherford, Bend, OR (US)

Correspondence Address: Doreen Rutherford, 56285 Stellar Dr., Bend, OR, 97707, US

(21) Appl. No.: 11/508,411

(22) Filed: Aug. 22, 2006

(51) Int. Cl. A61F 13/42

(52) U.S. Cl. 604/361

(57) ABSTRACT

In a reusable apparel undergarment having a body of absorbent textile, the body being divided into a stomach portion, which when in wear, is located on a wearer's stomach side and a back portion which, when in wear, is located on his back side, the stomach portion and the back portion being connected and fixed together at both side edge portions, respectively, of the stomach and back portions to form a pair of leg opening portions and a unitary waist opening portion, the waist opening portion and pair of leg opening portions being provided around entire peripheral edges thereof with elastic members forming substantially continuous gathers. The interior portion found in the center of the leg openings where the stomach portion and back portion meet contains the animal character graphic.

### BACKGROUND OF THE INVENTION

The present invention relates to a system and method for toilet training, and more particularly an article having interactive animal graphics providing a training aid.

The toilet training process incorporates a wide variety of different aspects, including many training techniques and training aids that may be used by parents and caregivers, hereinafter simply referred to as caregivers. The aspect of the total toilet training process is the change from diapers to using the toilet without fail. Training pants bridge the gap for the caregiver to teach the moment has begun to encourage toilet use, but caregiver instruction is limited when dealing with the simplicity of a child. Positive encouragement and reinforcement are two of the most widely used tools in the toilet training process but there is still much room for improvement. Specifically, caregivers are still searching for easier and quicker ways to guide their children successfully through the total toilet training process.

Many caregivers have difficulty in determining the readiness of a child to begin the toilet training process and underestimate the difficulty of communicating to young children. While various books, video tapes, charts with stickers, personalize toilets and interactive toilet training kits are available, there remains a need for new and improved communication and motivational mechanisms to facilitate the toilet training process. If a child does not respond to an initial toilet training instruction or introduction, not only can the caregiver be at a loss for finding the right method or tool to encourage the child, but the child may enter into a lower level of confidence. Thus, making the path to success a longer, uphill version.

### SUMMARY OF THE INVENTION

In response to the aforementioned deficiencies associated with prior systems introducing toilet use among youngsters a garment has been developed that provides toilet training aid benefits through the use of interactive animal graphics. The interactive graphic includes one or more permanent animal characters constructed on the inside of the garment where the wetness would occur. Particularly, when a child is given their “special” undergarment by a caregiver, the animal graphics provide an opportunity for teaching the negative effects of “wetting” on the animal object graphic. The permanent character graphic is illustrated in a sleeping position, in this way the article may permit the caregiver to interact with the child stressing the importance of not waking or disturbing the sleeping animal with wetness. Over time the garment serves as a device for preventing child incontinence and provides a method and reasoning for the use of the toilet.

Hence, in a reusable apparel, the invention concerns a lightweight undergarment having elasticized leg openings. The outer surface, a pleasant non-interactive print, the interior surface includes the permanent sleeping animal character graphic allowing open communication between the youngster and the caregiver.

### BRIEF DESCRIPTION OF THE DRAWINGS

FIG. 1 is a front view of the toddler panty.

FIG. 2 is a front view showing the inside of the toddler panty.

FIG. 3 is a top view of the toddler panty graphic after having been separated at the side seams and laid out flat.

### DESCRIPTION OF THE PREFERRED EMBODIMENT

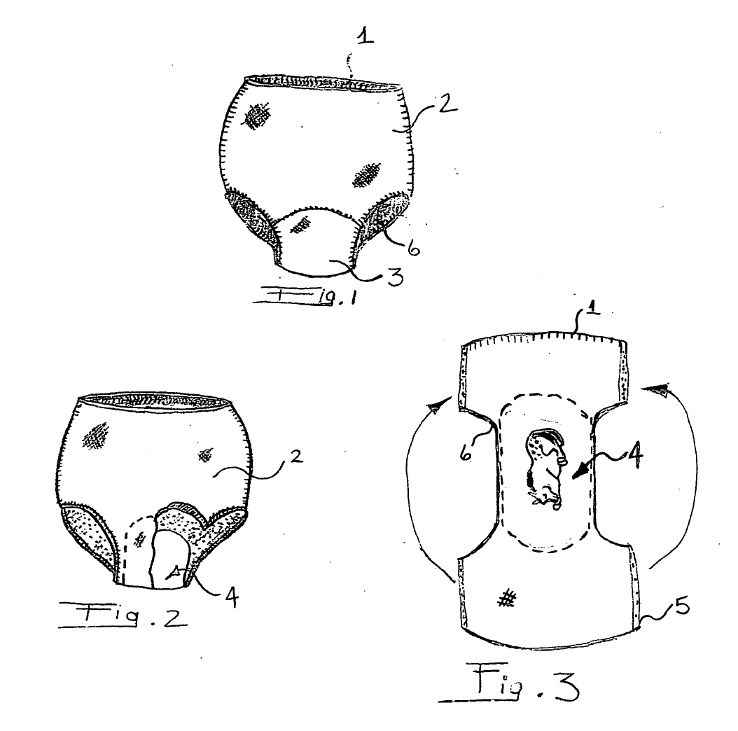

Referring now to the drawings in detail wherein like reference numerals represent like parts throughout the several figures, reference numeral 1 refers generally to the toddler panty. As shown in FIG. 1 the panty 1 has a soft cotton cloth 3 exterior and interior which provides a comfortable cushion between the toddlers skin and the training panty. The front 2 of the panty will resemble the back in texture and print. The legband area 6 will be perforated by a soft elastic for easy wear and will come in various sizes.

As shown in FIG. 2 and FIG. 3 the crotch area 4 is the location of the described graphic which allows communication to the toddler. An overlapping inner layer bearing the pony graphic and fabricated from a cotton washable substance or any appropriate material that provides the necessary zone of comfort is affixed to the inside of the toddler panty 4.

Referring now to FIG. 3 the sideseams 5 are a non separable seam joined by a chainstich which provides flexible movement and resembles an older childs undergarment.

In use and operation the graphic aided toddler panty is employed in exactly the same manner as a regular cloth training panty which is used in the transitional period from a diaper undergarment to normal child's panty. However, the graphic aided panty of the instant invention opens communication and instruction between the caregiver and the child with a simplistic goal. The construction of the toddler panty is very similar to that of the older child panty so that the child recognizes the difference in the transition from a training toddler disposable panty to a soft cotton child undergarment. Thus, the child can easily adapt to the new mode of behavior expected while wearing the special graphic.

What is claimed is:

1. An absorbent article comprising of a front part which contacts the wearers stomach during use;
a rear part which contacts the wearers buttocks during use;
a crotch part located between the wearers thighs during use and extends between said front and rear parts;
an absorbent body located on the crotch part which is enclosed between a liquid permeable material;
a single permanent character graphic disposed on the interior surface.

2. The absorbent article of claim 1, wherein the permanent character graphic illustrated is to be involved in an activity utilizing the single permanent character graphic as the subject of activity.

3. An absorbent article comprising:
an outer non-interactive plural graphic pattern surface having an opposite interior surface;
an absorbent assembly disposed on the interior surface;
a single permanent character graphic disposed on the inner surface;
wherein the permanent character graphic is interactively introduced through storyline to the wearer.

4. The absorbent article of claim 3, wherein the single permanent character graphic is located on the centerline of the interior surface as fore-mentioned in claim 1 as the crotch part.

5. The absorbent article of claim 4, wherein the absorbent article defines a longitude centerline and a transverse centerline, comprising of a plurality of graphics located on the exterior surface, and a single permanent character graphic located distinctly on the inner crotch region having a longitudinal direction;
wherein the single permanent character graphic has a size of at least about 3 by 2 centimeters and less than about 6 by 5 centimeters.
